(19) United States
(12) Patent Application Publication
(10) Pub. No.: US 2017/0292911 A1
(43) Pub. Date: Oct. 12, 2017

(54) APPARATUS AND METHOD FOR PROCESSING LIQUID SUBSTANCE FOR MEASUREMENT

(71) Applicant: VALMET AUTOMATION OY, Espoo (FI)

(72) Inventors: Pasi KÄRKI, Kajaani (FI); Tuomo TOMPERI, Kajaani (FI)

(73) Assignee: VALMET AUTOMATION OY, Espoo (FI)

(21) Appl. No.: 15/518,160

(22) PCT Filed: Oct. 23, 2015
(86) PCT No.: PCT/FI2015/050728

§ 371 Date: Apr. 10, 2017

(30) Foreign Application Priority Data
Oct. 23, 2014 (FI) 20145930

(51) Int. Cl. G01N 21/53; G01N 21/59; G01N 33/18; G01N 1/14

(52) U.S. Cl. CPC G01N 21/534; G01N 1/14; G01N 21/59; G01N 33/18; G01N 2001/1037

(57) ABSTRACT

An apparatus includes a piston cavity, a piston insertable in the piston cavity, and at least one flow cavity. Each flow cavity is inserted in the piston, alongside the piston cavity such that the flow cavity has a connection with the piston cavity, or between the piston and a wall of the piston cavity by means of one or more differences in cross section between the piston and the piston cavity, and the flow cavity is in contact with a liquid substance being processed. The piston moves back and forth inside the piston cavity and causes with its movement a flow of the liquid substance in each flow cavity in connection with measuring the liquid substance.

### FIELD

The invention relates to an apparatus and method for processing a liquid substance for measurement.

### BACKGROUND

The reject from centrifuges in a wastewater plant is measured such that the wastewater is sampled, and large particles and air are removed from the sample. Large particles in the sample are caused for instance by struvite, which crystallizes naturally in the slurry/onto the walls of wastewater process equipment. Struvite refers to a glasslike, crystallized substance. Struvite travels in pieces along with the wastewater and sticks to different parts of the process equipment. The removal of large particles is carried out by screening the sample. A screen, however, is easily clogged with solid matter, i.e. mainly struvite. Also sampling pipes become foul, particularly due to the influence of a struvite-containing sample. Screening also makes the sample foam, which interferes with the measurement of the reject and/or accept.

In wastewater plants, other embodiments requiring more advanced sampling include influent and effluent measurements. Measurement problems caused by mechanical fouling or chemical contamination of the sensors and sampling pipes used in the measurement commonly occur in mineral refining processes as well.

Therefore, a need exists for more sophisticated sampling for measurement of a liquid sample.

### BRIEF DESCRIPTION

An object of the invention is to provide an improved solution. This is achieved by an apparatus according to claim 1.

The invention also relates to a method according to claim 9.

Preferred embodiments of the invention are disclosed in the dependent claims.

The apparatus and method according to the invention provide several advantages. No separate screen-like filter is necessary, and it is possible to reduce the extent of solid dirt build-up or prevent it completely while taking and processing a sample, which enables a sampler to operate appropriately, an original sample to remain unchanged until being measured, and high-quality measurement to be provided. In addition, no need exists to separately return the sample, and no sample goes to waste down the drain to load the purification of water.

### LIST OF FIGURES

The invention is now described in closer detail in connection with the preferred embodiments and with reference to the accompanying drawings, in which

FIG. 1 shows an example of an apparatus for processing a liquid substance for measurement;

FIG. 2 shows an example of a situation wherein a piston of the apparatus is in a different position than in the case of FIG. 1;

FIG. 3 shows an example of an apparatus wherein a flow cavity is provided inside the piston,

FIG. 4 shows an example of an apparatus wherein the flow cavity and a piston cavity are parallel;

FIG. 5 shows an example of an apparatus wherein the flow cavity resides between a laterally sectioned piston and walls of the piston cavity, from a direction of a longitudinal axis of the piston cavity;

FIG. 6 shows an example of an apparatus wherein the flow cavity resides between the piston and the walls of a laterally expanded piston cavity, from the direction of the longitudinal axis of the piston cavity;

FIG. 7 shows an example of an apparatus wherein a sensor is provided in the flow cavity located inside the piston;

FIG. 8 shows an example of an apparatus wherein the sensor is provided in the flow cavity between the piston and the walls of the piston cavity;

FIG. 9 shows an exemplary flow chart of a method.

### DESCRIPTION OF EMBODIMENTS

The following embodiments are presented by way of example. Even though the description may refer to “an” embodiment or embodiments at different points, this does not necessarily mean that each such reference is made to the same embodiment or embodiments or that the feature only applies to one embodiment. Individual features of different embodiments may also be combined in order to enable other embodiments.

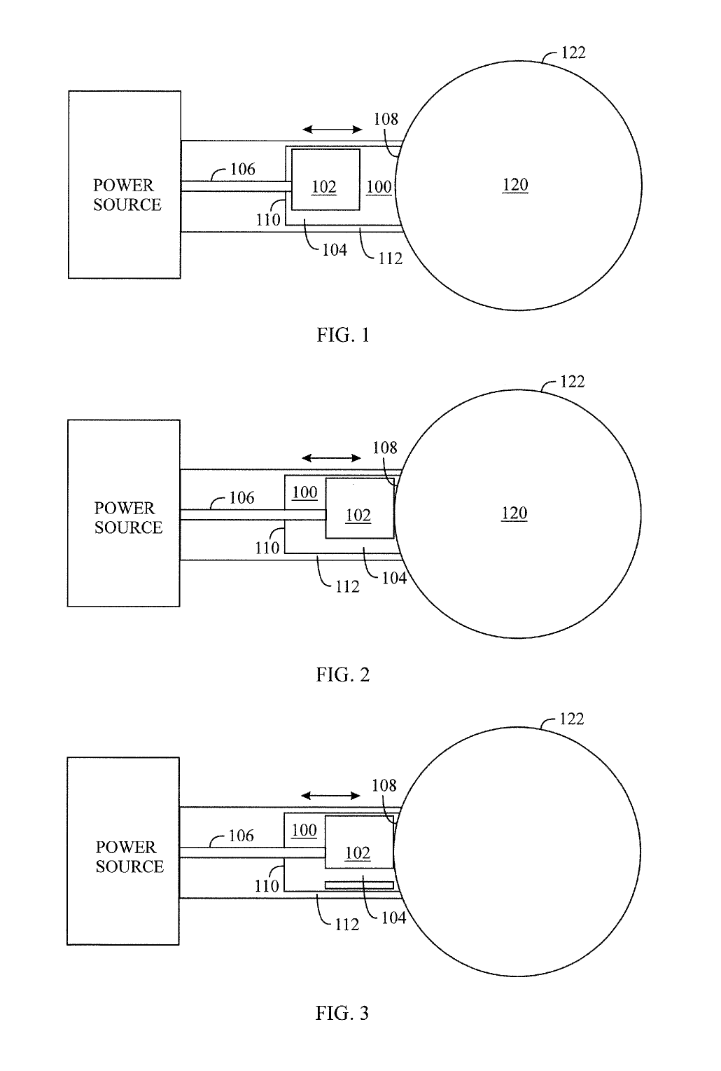

FIGS. 1 and 2 show an example of an apparatus for processing a liquid substance in connection with measurement. Such processing of a liquid substance may be necessary for measurement, for instance. In FIG. 1, showing a side view of the apparatus, a piston 102 is in its position farthest from the liquid substance 120 being sampled, or at least close to such a position. In this position, the piston 102 contacts or resides in the vicinity of an end 110. In an embodiment, the liquid substance 120 may be a suspension or the like. In an embodiment, the liquid substance 120 may be processed or unprocessed wastewater. In an embodiment, the liquid substance 120 may be stock used in paper industry, for instance. The apparatus comprises a piston cavity 100, a piston 102 insertable in the piston cavity 100, and at least one flow cavity 104. The piston cavity 100 is in contact with the liquid substance 120 being processed for measurement.

The apparatus may be coupled with a process pipe, reservoir or tank 122. The piston 102 with its movement receives the liquid substance 120 from the process pipe, reservoir or tank 122, and the piston 102 with its movement returns the liquid substance 120 back to the process pipe, reservoir or tank 122. In such a case, the movement of the piston 102 in one direction draws the liquid substance 120 from the process pipe, reservoir or tank 122 to the flow cavity 104 and/or to the piston cavity 100, and the movement of the piston 102 in the other direction pushes the liquid substance 120 contained in the flow cavity 104 and/or in the piston cavity 100 back to the process pipe 122. The apparatus thus takes the sample from the process pipe, reservoir or tank 122, and causes it to flow at least in the flow cavity 104 and optionally also in the piston cavity 100.

The cavity may be construed as a hole in a piece. The cavity may be for instance a cylinder or a pipe, wherein the walls of the cylinder or the pipe confine therein a cavity with a circular cross section, for instance. However, the cavity may be of any shape in its cross section.

At one of its ends 108, the piston cavity 100 may be open, and the open end 108 is in contact with the liquid substance 120 being processed for measurement, as shown in FIGS. 1 and 2. In such a case, the piston cavity 100 is directly connected with the liquid substance, and the liquid substance is allowed to flow through the open end 108 to the piston cavity 100. An opening in the open end 108 may be equal to or smaller than the piston cavity 100. In an embodiment, the liquid substance 120 may flow in the process pipe 122, for instance. In an embodiment, the liquid substance 120 may be contained in a reservoir or a tank being sampled. The process pipe 122 has an aperture so as to enable the liquid substance 120 to flow to the piston cavity and one or more flow cavities 100, 104.

At the other of its ends 110, the piston cavity 100 may be closed so as to prevent the liquid substance 120 from flowing through the end 110, as shown in FIGS. 1 and 2.

In FIGS. 1 and 2, the flow cavity 104 is located between the piston 102 and a wall 112 of the piston cavity 100 by means of one or more differences between the piston 102 and the piston cavity 100 in cross section. Such a difference in cross section may be for instance a difference in the shape of the cross section.

The piston 102 moves back and forth inside the piston cavity 100, causing with its movement a flow of the liquid substance 120 via each flow cavity 104 in to the piston cavity 100 and out of the piston cavity 100 for the measurement of suspension in said at least one flow cavity 104. The piston 102 moves in the piston cavity 100 in a direction of a longitudinal axis of the piston cavity 100, towards the liquid substance 120 and away from the liquid substance 120.

In an embodiment, the liquid substance 120 flows from between the piston cavity 100 and the piston 102, from one side of the piston 102 to the other side thereof, while the piston 102 moves.

In FIG. 2, showing a side view of the apparatus, the piston 102 is in its position closest to the liquid substance 120, or at least close to such a position. In this position, the piston 102 may in part or completely extend into the process pipe, reservoir or tank 122 containing the liquid substance 120.

The flow of the liquid substance 120 in the flow cavity 104 makes the flow reduce the extent of dirt build-up, enabling the flow cavity, piston cavity and/or one or more measurement sensors provided in the flow cavity 104 to stay clean. No air bubbles necessarily interfere with the measurement, either.

FIG. 3, a side view of the apparatus, shows a solution wherein the flow cavity 104 is inserted in the piston 102.

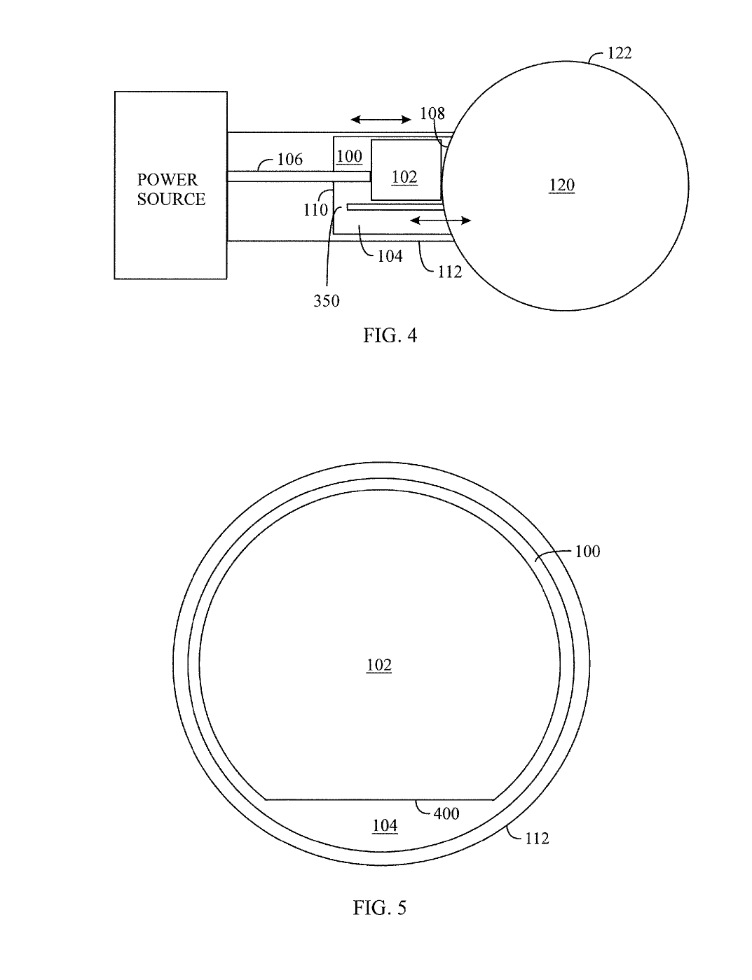

FIG. 4 shows a solution wherein the flow cavity 104 is parallel with the piston cavity 100. The flow cavity 104 is in an open connection with the piston cavity 100. When the piston 102 is in the open end 108 or closest to the process pipe, reservoir or tank 122, at least part of the piston 102 lies between the open connection 350 and the open end 108. The open connection 350 thus lies closer to the closed end 110 of the piston cavity 100 than to the open end 108 thereof. The open connection 350 may also abut on the closed end 110. Each flow cavity 104 is in contact with the liquid substance 120 being processed for measurement. The flow cavity 104 and the piston cavity 100 are thus coupled in parallel.

The piston 102 moves back and forth inside the piston cavity 100, causing with its movement a flow of the liquid substance 120 in each flow cavity 104. In this solution, the movement of the piston 102 produces a uniform flow of the liquid substance 120. It is advantageous for instance that the flow rate for wastewater, serving as the liquid substance 120, in the flow cavity 104 is more than 1 m/s or, preferably, more than 2 m/s. This applies to all embodiments disclosed in the present application. In connection with a centrifuge, the flow rate of wastewater in the flow cavity 104 may be more than 5 m/s. Such flow rates reduce fouling of the apparatus or even prevent the apparatus from being fouled with struvite or other dirt. The solution according to FIG. 4 in particular may be implemented as an in-line arrangement.

FIG. 5 shows an example of a piston cavity 100 and a piston 102, from a direction of the longitudinal axis of the piston cavity. In this example, the piston cavity 100 is a circle in cross section, but the cross section of the piston 102 is not a circle but one part 400 of an outer surface of the piston 102 is straight in accordance with a chord of the circular cross section. Thus, a flow cavity 104 is provided between the straight part 400 and the wall 112 of the piston cavity.

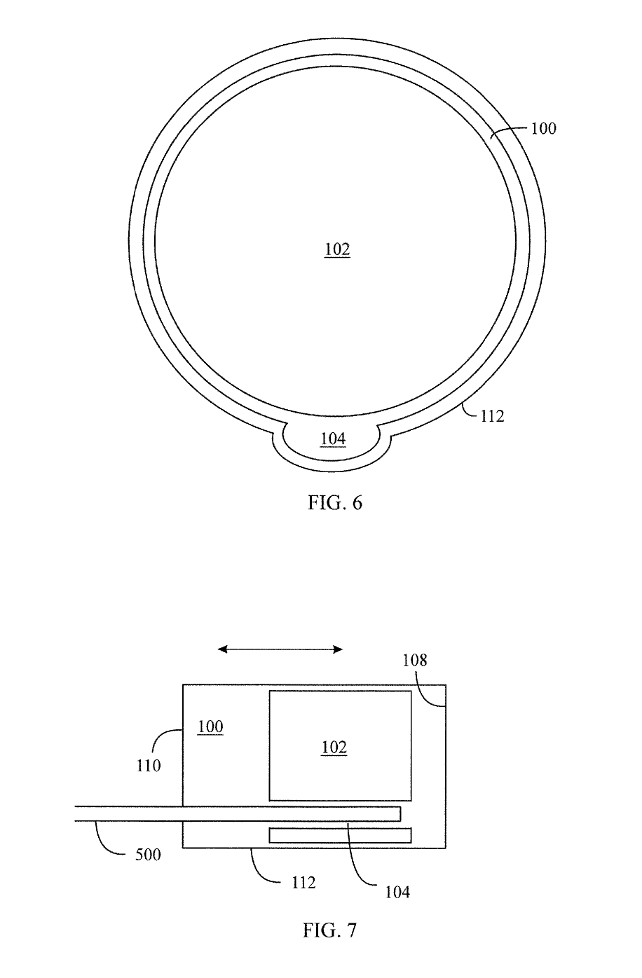

FIG. 6 shows another example of a piston cavity 100 and a piston 102, from the direction of the longitudinal axis of the piston cavity 100. In this example, the piston 102 is a circle in cross section, but the piston cavity 100 is provided with a protruding part which is formed by a protrusion in the wall 112 of the piston cavity 100 and which forms the flow cavity 104.

In an embodiment, the apparatus may comprise a piston rod 106. The piston rod 106 extends through an end 110 of the piston cavity 100 closed tight against the liquid substance 120. The rod 106 of the piston 102 is fixed to the piston 102 in order to move the piston by means of a longitudinal force exerted on the rod 106 of the piston 102. The rod 106 of the piston 102 may be moved by a power source which may generate motion for instance pneumatically, hydraulically or electrically.

In an embodiment, the piston 102 has no rod but the piston 102 is moved by means of an electric and/or magnetic field. The electric and/or magnetic field may be produced by a capacitive or inductive device. The inductive device may be an electromagnet. Also the piston 102 may comprise a magnet and/or an electromagnet. The capacitive or inductive device may be located at the edges, ends or in the vicinity of the ends of the piston cavity 100.

In an embodiment, the piston 102 is in its entirety arranged to stay inside the piston cavity 100 throughout its movement. In a general case, however, it may be that the piston 102 at least in part exits the inside of the piston cavity 100.

In an embodiment, shown in FIG. 7, a sensor 500 is insertable in the flow cavity 104 which may reside inside the piston 102.

In an embodiment, the sensor 500 is insertable in the flow cavity 104 which may reside between the piston 102 and the wall 112 of the piston cavity 100. The sensor 500, which may be rodlike or fibrelike, may be an optical fibre, for instance.

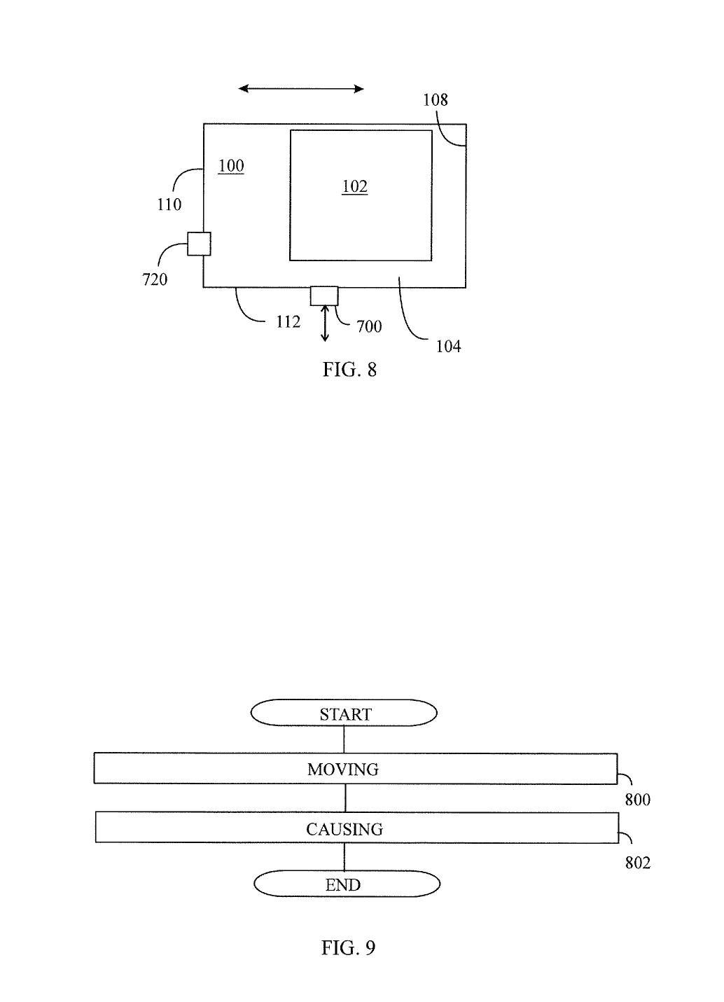

In an embodiment, shown in FIG. 8, a sensor 700 is insertable at least in part in the flow cavity 104 which may reside between the piston 102 and the wall 112 of the piston cavity 100. The sensor 700 may be any sensor, such as an optical sensor, electric sensor, magnetic sensor, acoustic sensor, mechanical sensor or the like. Optical, electric, magnetic or acoustic measurement may be a through-measurement comprising transmitting a signal through the liquid substance 120, or the measurement may be a reflection measurement comprising transmitting a signal to the liquid substance and receiving the signal reflected from the liquid substance. Such a through-measurement of the liquid substance 120 perpendicularly or at least almost perpendicularly to a direction of travel of the liquid substance 120 is possible in the solution according to FIG. 4 wherein the flow cavity 104 and the piston cavity 100 are parallel. The liquid substance may be measured for instance for consistency, density, gas concentration, viscosity, electrical conductivity, concentration of one or more substances, a combination thereof or the like.

In an embodiment, the apparatus comprises a detergent nozzle 720 enabling a detergent to be fed therethrough to an area between the piston 102 and the end 110 so as to keep the piston cavity 100, the piston 102, the flow cavity 104 and optionally also the sensor 500, 700 clean. The detergent may be base- or acid-based. The detergent may contain for instance deconex, hydrochloric acid, citric acid, chlorine, bromine or the like. In addition, detergents may be used by cycling, i.e. using them one after the other. In an embodiment, the cleaning may be carried out by ultrasound cleaning. The use of a detergent and the ultrasound cleaning may also be combined so as to enhance the cleaning result.

In an embodiment, the diameter of the piston cavity 100 is 10 mm to 100 mm, for instance. The piston 102 may be made to tightly fit the inside dimension of the piston cavity 100, or the piston 102 resides loosely in the piston cavity 100, in which case the liquid substance also flows somewhere else, not only in the flow cavity 104. Both the walls of the piston cavity 100 and the piston 102 may be made of metal, for instance. The metal may comprise for instance steel, copper or aluminium. Instead of metal, for instance a polymer or a ceramic may also be used.

FIG. 9 shows an example of a method. A method step 800 comprises moving a piston 102 back and forth in a piston cavity 100 connected at least with one flow cavity 104 which is in contact with a liquid substance 120 being processed. A step 802 comprises causing with the movement of the piston 102 a flow of the liquid substance 120 in said at least one flow cavity 104 inserted in the piston 102, alongside the piston cavity 100 such that the flow cavity 104 has a connection with the piston cavity 100, or between the piston 102 and a wall 112 of the piston cavity 100 by means of at least one difference in cross section between the piston 102 and the piston cavity 100, in connection with measuring the liquid substance 120.

Even though the invention has been described above with reference to the examples according to the accompanying drawings, it is clear that the invention is not restricted thereto but may be modified in many ways within the scope of the accompanying claims.

1-12. (canceled)

13. An apparatus for measuring a liquid substance containing particles, the apparatus comprising a sensor, a piston cavity, a piston insertable in the piston cavity, and at least one flow cavity, the apparatus being arranged to measure optically, electrically, magnetically or acoustically by the sensor the liquid substance, which contains stock, processed wastewater, unprocessed wastewater or a liquid sample taken from a mineral refining process, for consistency, density, gas concentration, viscosity, and/or electrical conductivity;
each flow cavity is inserted in the piston, alongside the piston cavity such that the flow cavity has a connection with the piston cavity, or which is between the piston and a wall of the piston cavity by means of one or more differences in the shape of the cross section between the piston and the piston cavity, and the flow cavity is in contact with the liquid substance being processed; and
the piston is arranged to move back and forth inside the piston cavity and to cause with its movement a flow of the liquid substance in each flow cavity in connection with measuring the liquid substance.

14. An apparatus as claimed in claim 13, wherein the piston cavity is in contact with the liquid substance being processed for measurement; and the piston is arranged to cause by its movement a flow of the liquid substance via each flow cavity in to the piston cavity and out of the piston cavity.

15. An apparatus as claimed in claim 13, wherein the piston cavity is at one of its ends open, and the open end is arranged to be in contact with the liquid substance being processed for measurement; and at its other end the piston cavity is closed.

16. An apparatus as claimed in claim 13, further comprising a piston rod, and through the end of the piston cavity closed tight against the liquid substance is arranged to extend the rod of the piston fixed to the piston in order to move the piston by means of a longitudinal force exerted on the rod of the piston.

17. An apparatus as claimed in claim 13, wherein the piston is in its entirety arranged to stay inside the piston cavity throughout its movement.

18. An apparatus as claimed in claim 13, wherein the sensor is at least in part insertable in the flow cavity.

19. An apparatus as claimed in claim 13, further comprising at least one measurement sensor for measuring the liquid substance.

20. An apparatus as claimed in claim 13, wherein the apparatus is connectable with a process pipe, reservoir or tank, and the piston is arranged with its movement to receive the liquid substance from the process pipe, reservoir or tank and to return the liquid substance back to the process pipe, reservoir or tank.

21. A method for measuring a liquid substance containing particles, the method comprising:
moving a piston back and forth in a piston cavity connected at least with one flow cavity which is in contact with the liquid substance being processed;
measuring optically, electrically, magnetically or acoustically by a sensor the liquid substance, which contains stock, processed wastewater, unprocessed wastewater or a liquid sample taken from a mineral refining process, for consistency, density, gas concentration, viscosity, and/or electrical conductivity; and
causing with the movement of the piston a flow of the liquid substance in said at least one flow cavity inserted in the piston, alongside the piston cavity such that the flow cavity has a connection with the piston cavity, or between the piston and a wall of the piston cavity by means of at least one difference in the shape of the cross section between the piston and the piston cavity, in connection with measuring the liquid substance.

22. A method as claimed in claim 21, further comprising moving the piston through an end of the piston cavity closed tight against the liquid substance by a rod of the piston by means of a longitudinal force exerted thereon.

23. A method as claimed in claim 21, further comprising the piston staying in its entirety inside the piston cavity throughout its movement.

24. A method as claimed in claim 21, further comprising the sensor being at least in part inserted in the flow cavity.
